(19) United States
(12) Patent Application Publication
(10) Pub. No.: US 2013/0040991 A1
(43) Pub. Date: Feb. 14, 2013

(54) PHARMACEUTICAL COMPOSITION COMPRISING GEMFIBROZIL AND CYP2C8 AND/OR OATP SUBSTRATE DRUG SUCH AS REPAGLINIDE

(71) Applicants: Pertti Neuvonen, Helsinki (FI); Mikko Niemi, Helsinki (FI); Janne Backman, Espoo (FI)

(72) Inventors: Pertti Neuvonen, Helsinki (FI); Mikko Niemi, Helsinki (FI); Janne Backman, Espoo (FI)

(21) Appl. No.: 13/637,519

(22) PCT Filed: Mar. 29, 2011
(86) PCT No.: PCT/FI11/50265

§ 371 Date: Oct. 16, 2012

(30) Foreign Application Priority Data
Mar. 30, 2010 (FI) 20105327

(51) Int. Cl. A61K 31/192; A61P 3/10; A61P 3/06; A61K 31/451

(52) U.S. Cl. 514/331; 514/571

(57) ABSTRACT

The invention provides gemfibrozil for use in adjusting the effect of a CYP2C8 and/or OATP substrate drug, wherein gemfibrozil is administered in an amount of less than 1200 mg/day. The invention also provided a pharmaceutical composition comprising gemfibrozil providing a significant improvement to the plasma profiles of a CYP2C8 and/or OATP substrate drug in a mammal.

### FIELD OF THE INVENTION

The invention relates to the field of pharmaceuticals and in particular to a pharmaceutical composition comprising gemfibrozil. Gemfibrozil or the metabolite thereof inhibits the activities of cytochrome P450 (CYP) 2C8 isozyme and organic anion transporting polypeptides (OATP).

### BACKGROUND OF THE INVENTION

Cytochrome P450 (CYP) is a superfamily of enzymes responsible for metabolism of most drugs and many endogenous substances in living organisms. CYP2C8 is one of the CYP isoenzymes but its role in the metabolism of many drugs has not been recognized until recent years. Even today, there is insufficient and incorrect information on the role of CYP2C8 in the metabolism of many drugs.

CYP2C8 is known to be important to the metabolism of several drugs including, for example, certain orally used diabetes drugs and certain anti-cancer agents. The activity of CYP2C8 varies genetically, which can cause significant interindividual variation in the plasma concentrations, effects and risk of adverse effects of CYP2C8 substrate drugs. CYP2C8 is sensitive also for certain drug interactions.

Different OATPs can affect the transport of their substrate drugs into cell, e.g. into liver cells. Gemfibrozil and its glucuronide metabolite can inhibit the activity of these transporters and affect the pharmacokinetics, efficacy and risk of adverse effects of their substrate drugs.

The adverse effects of drugs can cause severe problems in many patients. Many of these adverse effects are concentration-dependent, generally called as type A adverse effect. Prediction of correct drug response is difficult if there is large inter-individual variation in plasma drug concentrations, due to, e.g., genetic variation in the activity of drug metabolizing enzymes or transporters. This variability increases the risk of adverse effects because drugs are usually dosed according to the needs of an “average” patient. Thus, there is a risk of too high drug concentrations (increased toxicity) or too low drug concentration (insufficient efficacy) in individual patients. Individual variation can cause several-fold differences in plasma drug concentrations between different subjects, which can cause serious over- or under-treatment, particularly, if the drug has a narrow therapeutic index (small margin between the effective and toxic concentrations).

Optimal plasma profiles of drugs depend on the drug in question and on the indication, to which the drug is aimed. A short-lasting, high peak plasma concentration of a drug can be optimal depending on its mechanism of action and indication. On the other hand, low peak concentration and slow rate of elimination are preferable if the peak concentrations cause adverse effects, or a prolonged, flat plasma concentration is needed to the optimal effect. This is particularly important in continuous drug therapy, because longer half-life allows a less frequent dosing of a drug, which can be an important advantage also to the efficacy. Furthermore, an increase in oral bioavailability, e.g. in the rate or extent of absorption, decreases the geneticallybased inter-individual variation in drug response and the risk of many drugdrug interactions.

Gemfibrozil, or 5-(2,5-dimethylphenoxy)-2,2-dimethyl-pentanoic acid, is a drug commonly used for lowering lipid levels and as such used in treatment of hyperlipidemia. Gemfibrozil in a daily dose of 1 200 mg has been in the long-term clinical use for the treatment of hyperlipidemias, particularly for triglyceridemias, for more than 20 years and its safety has been well documented in many clinical trials, including Helsinki Heart Study. However, gemfibrozil has greatly increased the risk of serious adverse effects, including deaths, when used simultaneously with certain other drugs (e.g. cerivastatin). The increased toxicity of cerivastatin in the concomitant use with gemfibrozil was due to adverse drug interactions mediated by inhibition of CYP2C8 and/or OATP transporters. This interaction led to muscle-toxic plasma concentrations of the CYP2C8 substrate drug cerivastatin (Backman J T, Kyrklund C, Neuvonen M, Neuvonen P J., Clin Pharmacol Ther. 2002 December; 72(6):685-91).

A concomitant use of gemfibrozil and the antidiabetic drug repaglinide has also caused serious adverse effects (hypoglycaemias), and their combined use has been contradicated by the European Medicines Agency. The mechanism of this life-threatening adverse effect is inhibition of CYP2C8-mediated metabolism of repaglinide and/or inhibition of OATPmediated transport of repaglinide by gemfibrozil and/or its glucuronide (Niemi et al, Effects of gemfibrozil, itraconazole and their combination on the pharmacokinetics and pharmacodynamics of repaglinide: potentially hazardous interaction between gemfibrozil and repaglinide; Diabetologia (2003); 46:347-351).

It is known that the parent gemfibrozil is relatively weak inhibitor of CYP2C8 but its main metabolite formed in vivo, i.e. gemfibrozil 1-O-β-glucuronide, is a potent inhibitor and inactivator of CYP2C8. The risk of serious gemfibrozil-related interactions persists at least for the 12 hours' dosing interval during the usual daily dosing of 600 mg gemfibrozil twice daily (Tornio et al, The effect of Gemfibrozil on Repaglinide Pharmacokinetics Persists for at Least 12 h After the Dose: Evidence for Mechanism-based Inhibition of CYP2C8 In Vivo, Clin Pharmacol Ther. 2008 September, 84(3): 403-411). The activity of CYP2C8 gradually recovers in 1 to 4 days after the last dose of daily administration of gemfibrozil 600 mg twice daily (Backman et al., CYP2C8 Activity Recovers within 96 hours after Gemfibrozil Dosing: Estimation of CYP2C8 Half-Life Using Repaglinide as an in Vivo Probe, Drug Metab Dispos. 2009; 37(12) 2359-2366).

The concomitant use of gemfibrozil and certain CYP2C8 substrate drugs, such as repaglinide, is contraindicated by the European Medicines Agency and the Food and Drug Administration (FDA). Thus, based on the present public knowledge, the simultaneous use of gemfibrozil with such drugs, which are significantly metabolized by CYP2C8, should be avoided or is even contraindicated.

### DEFINITIONS

As used herein, the term “gemfibrozil” is meant to include the drug gemfibrozil, gemfibrozil-1-O-β-glucuronide or any prodrug which is capable of being metabolized into gemfibrozil-1-O-β-glucuronide, if not otherwise stated. In an embodiment of the invention, “gemfibrozil” means gemfibrozil drug itself. In another embodiment, “gemfibrozil” means gemfibrozil-1-O-β-glucuronide, or any prodrug which is capable of being metabolized into gemfibrozil-1-O-β-glucuronide.

As used herein, the term “CYP2C8 substrate drug” is meant to include any currently known or unknown pharmacologically active compound or prodrug which is metabolized by CYP2C8 and/or transported by OATP transporters.

### BRIEF DESCRIPTION OF THE INVENTION

The inventors have now surprisingly found that the plasma profiles of CYP2C8 and/or OATP1B1 substrate drugs can be optimized according to different clinical needs by changing the ratio or amount of gemfibrozil, or of gemfibrozil and of the substrate drugs in a pharmaceutical formulation.

A typical daily dose of gemfibrozil in its normal use for treating lipid disorders is 1 200 mg administered in two equal doses of 600 mg each. It was surprisingly found that gemfibrozil works as indicated above as enhancer of various CYP2C8 and/or OATP substrate drugs intended for various indications already in very small, even less than 10% of its usual clinically used doses.

In an aspect, the invention provides gemfibrozil for use in adjusting the effect of a CYP2C8 and/or OATP substrate drug, wherein gemfibrozil is administered in an amount of less than 1 200 mg/day to a mammal. In an embodiment of the invention, the mammal is a human.

In another aspect, the invention provides a use of gemfibrozil for adjusting the effect of a CYP2C8 and/or OATP substrate drug, wherein gemfibrozil is administered in an amount of less than 1 200 mg/day.

In still another aspect, the invention provides a pharmaceutical composition which increases the efficacy, safety and predictability of effect of CYP2C8 and/or OATP substrate drugs. This is particularly important for drugs which have a narrow therapeutic index, such as oral antidiabetic drugs and anticancer drugs. By varying the relative amounts of gemfibrozil and the CYP2C8-subsrate drugs in the pharmaceutical formulation it is possible to alter the peak concentration, bioavailability, or rate of elimination of the CYP2C8 substrate drugs and/or to decrease daily fluctuation of their plasma concentration, and or to reduce the formation of their toxic metabolites.

In a further aspect, the invention provides a method for adjusting the effect of a CYP2C8 and/or OATP substrate drug in a mammal, comprising administering a CYP2C8 and/or OATP substrate drug to the mammal, and administering gemfibrozil to the mammal in an amount of less than 1 200 mg/day.

In an even further aspect, the invention provides a method of improving the plasma profiles of a CYP2C8 and/or OATP substrate drug in a mammal, comprising administering a CYP2C8 and/or OATP substrate drug to the mammal, and administering gemfibrozil to the mammal in an amount of less than 1 200 mg/day.

A specific advantage of the invention is that gemfibrozil can be used in small amounts having no clinically meaningful effect on lipid levels in a subject. Probability of any adverse effects caused by gemfibrozil in a subject can thus be minimized in a desired manner. Moreover, administration of unnecessarily high amounts of gemfibrozil to a subject having no problems with too high lipid levels can be avoided. It is thus highly desirable to provide pharmaceutical compositions/formulations for oral administration containing small amounts of gemfibrozil. These compositions can be easily combined with various substrate drugs in an appropriate manner for adjusting the effect of the substrate drug. However, also doses of gemfibrozil influencing blood lipids can be included.

Another advantage of the invention is that the doses of CYP2C8 substrate drugs in the formulations can be reduced due to the improved oral bioavailability, or to decreased rate of elimination, providing thus significant cost savings.

A further advantage is that formation of possibly toxic metabolites of CYP2C8 substrate drugs can be prevented by adding gemfibrozil to the pharmaceutical composition.

### BRIEF DESCRIPTION OF THE DRAWINGS

FIG. 1A shows the effect of various oral doses (0 mg, 30 mg, 100 mg, 300 mg and 900 mg) of gemfibrozil on plasma concentrations of a CYP2C8 and OATP substrate drug (repaglinide 0.25 mg). Mean plasma concentrations of repaglinide in six human subjects are given.

FIG. 1B shows the same data as illustrated in FIG. 1A on semi logarithmic scale to demonstrate the effect on elimination phase (half-life) of repaglinide.

FIG. 1C shows the mean plasma concentrations of gemfibrozil and gemfibrozil 1-O-β-glucuronide after an oral dose of 30 mg, 100 mg, 300 mg and 900 mg of gemfibrozil following an overnight fast in six healthy volunteers.

FIG. 2A shows the estimated effects of different doses of gemfibrozil (0 mg, 30 mg, 100 mg, 300 mg and 900 mg) on plasma profiles of repaglinide.

FIG. 2B shows the same data on semi logarithmic scale to demonstrate the effect on elimination phase (half-life).

FIG. 3A shows the effect of gemfibrozil (600 mg=black circles, 100 mg=stars, or 0 mg=open circles) given twice daily on plasma concentrations of four different CYP2C8/OATP substrate drugs (simvastatin, loperamide, rosiglitazone and pioglitazone).

FIG. 3B shows the same data on semi logarithmic scale to demonstrate the effect on elimination phase (half-life).

FIG. 4 shows the mean fold increase in the AUC (AUCᵢ/AUC_{c}) of repaglinide with different gemfibrozil doses (30 mg, 100 mg, 300 mg and 900 mg) in relation to the mean average plasma concentration of gemfibrozil 1-O-β-glucuronide during 10 hours after gemfibrozil dosing (C_{avg}) in six human subjects.

### DETAILED DESCRIPTION OF THE INVENTION

The present invention provides gemfibrozil for use in adjusting the pharmacokinetics and/or effect of a CYP2C8 and/or OATP substrate drug, wherein gemfibrozil is administered in an amount of less than 1 200 mg/day. In an embodiment of the invention, gemfibrozil is administered in an amount of less than 900 mg/day, preferably at most 300 mg/day, more preferably at most 200 mg/day, even more preferably at most 100 mg/day, specifically at most 30 mg/day.

In another aspect, the invention provides a use of gemfibrozil for adjusting the pharmacokinetics and/or effect of a CYP2C8 and/or OATP substrate drug, wherein gemfibrozil is administered in an amount of less than 1 200 mg/day. In an embodiment of the invention, gemfibrozil is administered in an amount of less than 900 mg/day, preferably at most 300 mg/day, more preferably at most 200 mg/day, even more preferably at most 100 mg/day, specifically at most 30 mg/day.

In a further aspect, the invention provides a method for adjusting the effect of a CYP2C8 and/or OATP substrate drug in a mammal, comprising administering a CYP2C8 and/or OATP substrate drug to the mammal, and administering gemfibrozil to the mammal in an amount of less than 1 200 mg/day. In an embodiment of the invention, gemfibrozil is administered in an amount of less than 900 mg/day, preferably at most 300 mg/day, more preferably at most 200 mg/day, even more preferably at most 100 mg/day, specifically at most 30 mg/day.

In the context of the present invention, the term “adjusting the effect of a CYP2C8 and/or OATP substrate drug”, includes optimizing plasma profile and/or pharmacokinetics of the substrate drug, enhancing the therapeutic efficacy of the substrate drug, preventing adverse and/or side-effects of the substrate drug, and preventing and/or inhibiting the formation of toxic metabolites of the substrate drug.

Accordingly in one embodiment, the invention relates to gemfibrozil for use in optimizing plasma profile and/or pharmacokinetics of a CYP2C8 and/or OATP substrate drug, wherein gemfibrozil is administered in an amount of less than 1 200 mg/day. In another embodiment, the invention relates to gemfibrozil for use in enhancing the therapeutic efficacy of a CYP2C8 and/or OATP substrate drug, wherein gemfibrozil is administered in an amount of less than 1 200 mg/day. In a further embodiment, the invention relates to gemfibrozil for use in preventing adverse and/or side-effects of a CYP2C8 and/or OATP substrate drug, wherein gemfibrozil is administered in an amount of less than 1 200 mg/day. In even further embodiment, the invention relates to gemfibrozil for use in preventing and/or inhibiting the formation of toxic metabolites of a CYP2C8 and/or OATP substrate drug, wherein gemfibrozil is administered in an amount of less than 1 200 mg/day.

Further, in one embodiment the invention relates to use of gemfibrozil in optimizing plasma profile and/or pharmacokinetics of a CYP2C8 and/or OATP substrate drug, wherein gemfibrozil is administered in an amount of less than 1 200 mg/day. Another embodiment of the invention relates to use of gemfibrozil in enhancing the therapeutic efficacy of a CYP2C8 and/or OATP substrate drug, wherein gemfibrozil is administered in an amount of less than 1 200 mg/day. A further embodiment of the invention relates to use of gemfibrozil in preventing adverse and/or side-effects of a CYP2C8 and/or OATP substrate drug, wherein gemfibrozil is administered in an amount of less than 1 200 mg/day. And an even further embodiment of the invention relates to use of gemfibrozil in preventing and/or inhibiting the formation of toxic metabolites of a CYP2C8 and/or OATP substrate drug, wherein gemfibrozil is administered in an amount of less than 1 200 mg/day.

In a still further embodiment, the invention relates to a method for optimizing plasma profile and/or pharmacokinetics of a CYP2C8 and/or OATP substrate drug in a mammal, wherein gemfibrozil is administered to the mammal in an amount of less than 1 200 mg/day. In another embodiment, the invention relates to a method for enhancing the therapeutic efficacy of a CYP2C8 and/or OATP substrate drug, wherein gemfibrozil is administered in an amount of less than 1 200 mg/day. A further embodiment of the invention relates to a method for preventing adverse and/or side-effects of a CYP2C8 and/or OATP substrate drug, wherein gemfibrozil is administered in an amount of less than 1 200 mg/day. Still a further embodiment of the invention relates to a method for preventing and/or inhibiting the formation of toxic metabolites of a CYP2C8 and/or OATP substrate drug, wherein gemfibrozil is administered in an amount of less than 1 200 mg/day.

In another aspect, the invention provides a pharmaceutical composition comprising gemfibrozil and a CYP2C8 and/or OATP substrate drug provided that the amount of gemfibrozil is not 200 mg and the amount of atorvastatin is not 40 mg.

Gemfibrozil, and particularly its metabolite gemfibrozil-1-O-β-glucuronide, are capable of inhibiting the activity of CYP2C8 isozyme and/or OATP transporters, e.g. OATP1B1, in vivo. The CYP2C8 isozyme and OATP transporters are actively participating in the metabolism and transport of drugs and have an impact on the absorption, plasma concentration and elimination rate of the drug.

The composition of the invention comprises gemfibrozil in an amount of 0.5 mg-100 mg/dose, preferably 1-100 mg/dose, more preferably 30-100 mg/dose, providing a significant improvement to the plasma profiles of a CYP2C8 and/or OATP substrate drug in a mammal. When gemfibrozil-1-O-β-glucuronide or a prodrug which is capable of being metabolized into gemfibrozil-1-O-β-glucuronide is used in the composition, the amounts thereof are calculated as gemfibrozil.

The amount of the CYP2C8 and/or OATP substrate drug in the formulation is then adjusted depending on the properties of said substrate drug. Need of delivering the composition to one or more dosages, and the dosage frequency per day are determined depending on the properties of the specific substrate drug and a condition to be treated. Typically, the amounts of the CYP2C8 and/or OATP substrate drugs vary from 0.1 mg to 1 000 mg but may be even less than 0.1 mg and more than 1 000 mg, depending on the potency of drugs and their indications. CYP2C8 substrate drugs belong to diverse therapeutic groups, e.g. oral antidiabetic drugs, cholesterol lowering drugs, and anti-cancer drugs.

The pharmacologically active compound, i.e. the CYP2C8 substrate drug, may be a single pharmacologically active molecule, or a combination of pharmacologically active molecules. CYP2C8 substrate drugs include, but are not limited, to the following drugs:
repaglinide, rosiglitazone, pioglitazone, troglitazone, montelukast, zafirlukast, pranlukast, paclitaxel, all-trans-retinoic acids, amiodarone, amodiaquine, chloroquine, piperaquine, imatinib, nilotinib, lapatinib, diltiazem, verapamil, loperamide, silybin, diclofenac, bexarotene, tazarotenic acid, cyclophosphamide, simvastatin, lovastatin, atorvastatin, cerivastatin, fluvastatin and rosuvastatin.

In an embodiment of the invention, the composition comprises, e.g., 100 mg of gemfibrozil and 0.25 mg of repaglinide. In another embodiment, the composition comprises 30 mg of gemfibrozil and 0.25 mg of repaglinide.

In still another object, the invention provides a pharmaceutical composition comprising gemfibrozil in amount of less than 300 mg, preferably about 200 mg, more preferably about 100 mg, even more preferably about 30 mg. In an embodiment, gemfibrozil is the single therapeutically active agent in the composition.

In a further aspect, the invention provides a pharmaceutical composition comprising gemfibrozil and a CYP2C8 and/or OATP substrate drug, whereby formation of toxic metabolites of the CYP2C8 substrate drug is prevented.

Gemfibrozil used in the amounts given in the present invention in pharmaceutical formulations helps optimization of the plasma profiles of CYP2C8 and/or OATP-substrate drugs. In context of the present invention, the term “optimization of the plasma profiles” has a meaning of increased bioavailability, decreased rate of elimination, decreased daily fluctuation in plasma drug concentrations, increased peak plasma concentration, and/or decreased variation in drug concentration or effects between subjects.

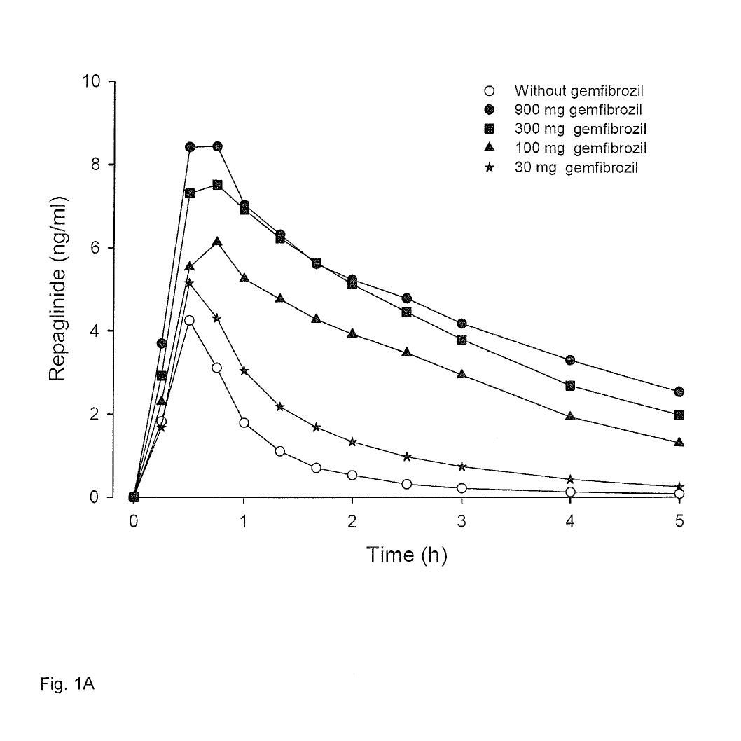

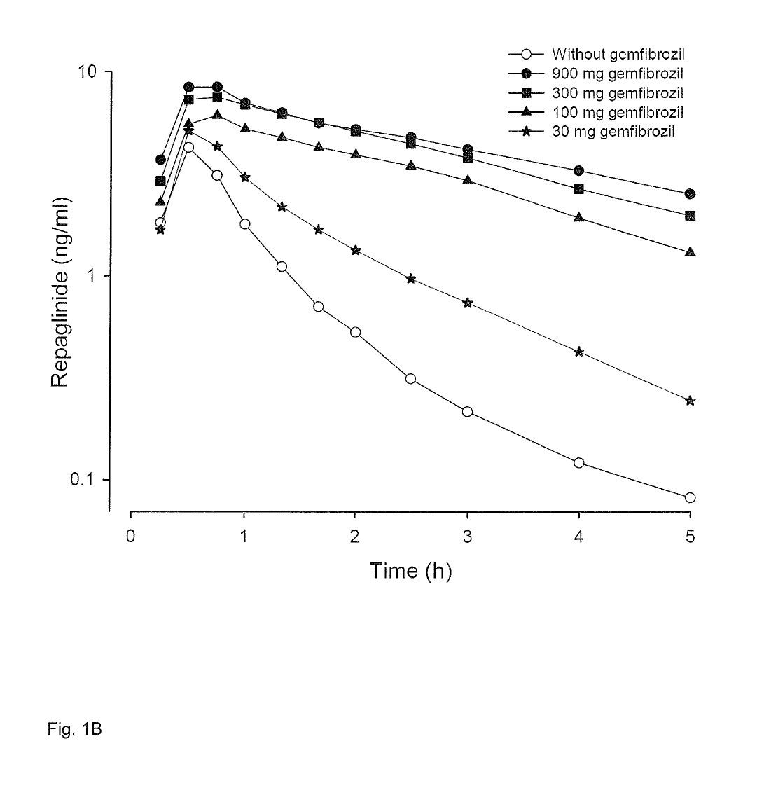

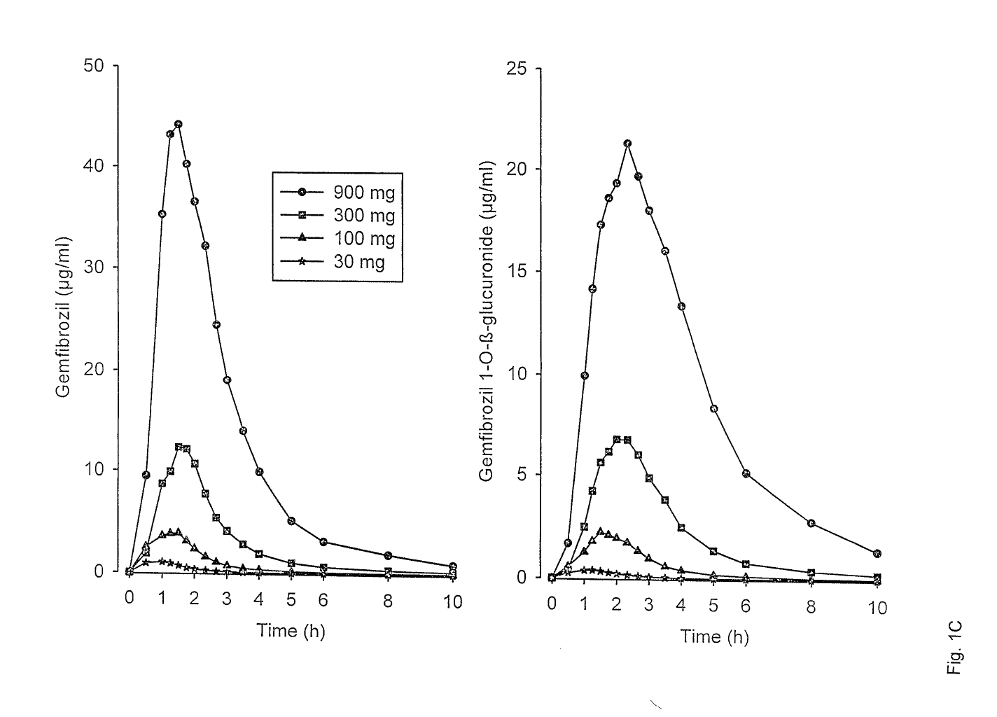

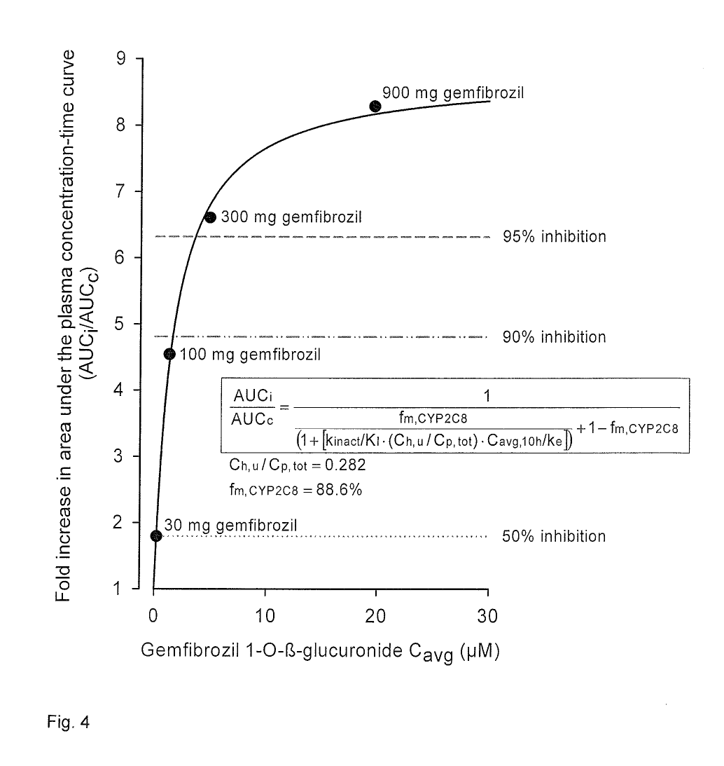

As stated above, gemfibrozil in its usual doses has caused serious problems when used simultaneously with the substrate drugs of CYP2C8 and OATP, and their simultaneous use is not recommended or it is even contraindicated by European Medicines Agency and by pharmaceutical companies. The inventors of the current invention have now surprisingly found that gemfibrozil can be used for optimizing the plasma concentrations and/or improving the bioavailability (FIGS. 1A, 1B, 2A, 2B, 3A, 3B, and 4) of drugs normally metabolised by the CYP2C8 isozyme, when gemfibrozil and/or CYP2C8 substrate drugs are used at the reduced doses compared to the generally used doses. FIG. 4 further shows a steep concentration-effect relationship with small gemfibrozil doses, such as 100 mg. Even smaller doses of gemfibrozil of 30 mg or less in repeated dosing can be used.

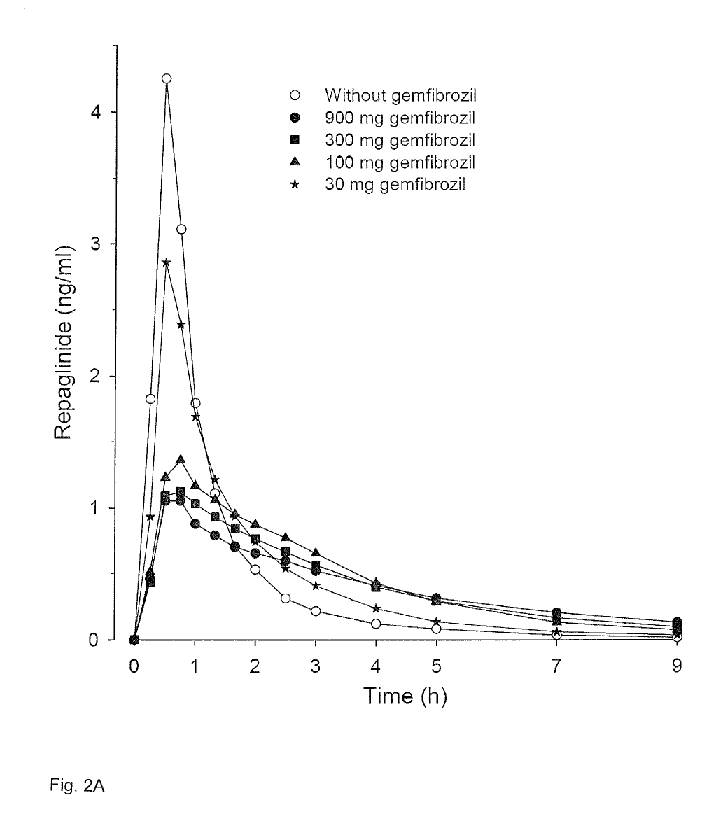

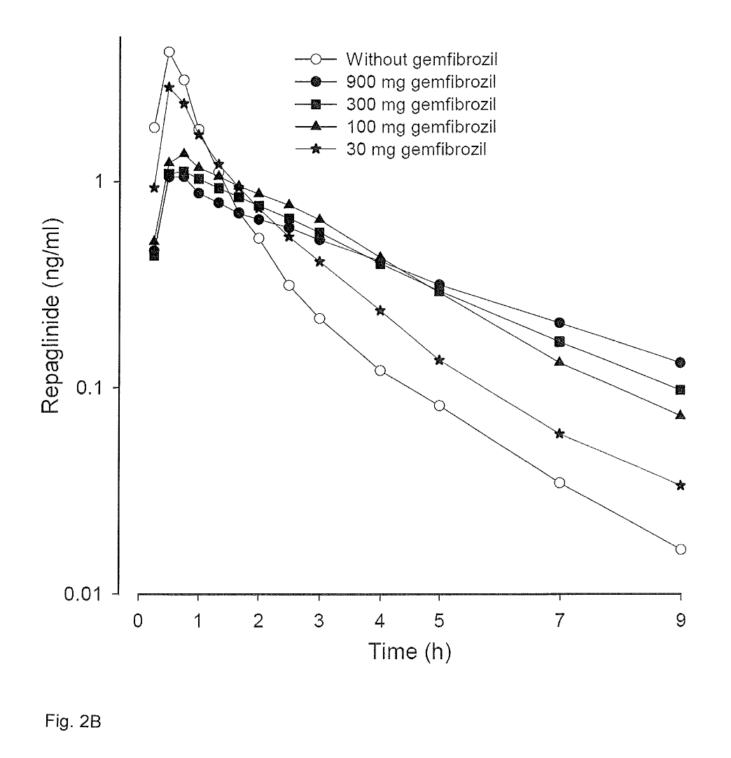

The improvement of the bioavailability of the CYP2C8 substrate drug includes a more predictable, prolonged and constant plasma concentration as well as prolonged half-life of the elimination of the drug. Thus the administered amount of CYP2C8 substrate drug can be significantly reduced from that currently used. By adjusting the administered amount of gemfibrozil and CYP2C8 substrate drugs, different bioavailability and elimination patterns (FIGS. 2A, 2B) and properties can be achieved for different purposes, depending on the optimal plasma profiles of the CYP2C8 substrate drug in question.

The compositions of the current invention are not limited to a certain formulation, instead the composition can be formulated into any known pharmaceutically acceptable formulation including but not limited to oral, enteral (e.g. rectal), and also to parenteral routes of administration. In an embodiment, the compositions of the invention are not intended for intrajejunal administration. In another embodiment, the composition of the invention is in an oral dosage form, such as, but not limited to, a tablet, capsule or a mixture.

The compositions according to the current invention can further comprise pharmaceutically acceptable adjuvants, additives, carriers and/or excipients. Selection of suitable ingredients and appropriate manufacturing methods for formulating the compositions belongs to general knowledge of a man skilled in the art. The compositions can be formulated in a sustained release formulation or an instant release formulation form.

In an embodiment of the invention, gemfibrozil is formulated in a first formulation and the CYP2C8 and/or OATP substrate drug is formulated in a second formulation, and the first and the second formulations are administered simultaneously or sequentially, in any order, to a mammal. In an embodiment, gemfibrozil is administered prior to administration of the substrate drug. Administration of the substrate drug can take place within 48 hours, preferably 24 hours, from the administration of gemfibrozil. In another embodiment, the substrate drug is administered prior to administration of gemfibrozil. This may be appropriate, for example, when it is desirable to prolong the elimination half-life of the substrate drug without increasing its peak concentration.

In another embodiment of the invention, gemfibrozil and the substrate drug are formulated in the same single formulation.

In a still further aspect, the invention provides a method of improving the plasma profiles of a CYP2C8 and/or OATP substrate drug in a mammal, comprising administering a CYP2C8 and/or OATP substrate drug to a mammal, and administering gemfibrozil to a mammal in an amount of less than 1 200 mg/day.

### Example 1

The effect of various oral doses (0 mg, 30 mg, 100 mg, 300 mg and 900 mg capsules) of gemfibrozil on plasma concentrations of a CYP2C8 and OATP1B1 substrate drug (repaglinide) was measured. Mean plasma concentrations of repaglinide in six human subjects are given. The subjects ingested in a cross-over study the same small dose (0.25 mg) of repaglinide with different doses of gemfibrozil. Venous blood samples were taken at fixed time points and plasma repaglinide concentrations were determined using a validated liquid chromatographic-tandem mass spectrometric (LC/MS-MS) method. The results are shown in FIG. 1A. FIG. 1B describes the same data on semi logarithmic scale to demonstrate the effect on elimination phase (half-life) of repaglinide.

The mean plasma concentrations of gemfibrozil and gemfibrozil 1-O-β-glucuronide after an oral dose of 30 mg, 100 mg, 300 mg and 900 mg of gemfibrozil following an overnight fast in six healthy volunteers are shown in FIG. 1C.

### Example 2

The effects of different doses of gemfibrozil (0 mg, 30 mg, 100 mg, 300 mg and 900 mg) on plasma profiles of repaglinide were measured. The doses of repaglinide were adjusted so that the total exposure to repaglinide (defined as the area under plasma repaglinide concentration-time curve, AUC) is the same in all 5 cases, corresponding to the AUC after a single dose of 0.25 mg repaglinide without gemfibrozil. The estimated peak concentration of repaglinide is reduced and its elimination half-life (and effect) is prolonged when the ratio of gemfibrozil to the CYP2C8 substrate drug (repaglinide) is increased. The results are shown in FIG. 2A. FIG. 2B describes the same data on semi logarithmic scale to demonstrate the effect on elimination phase (half-life).

### Example 3

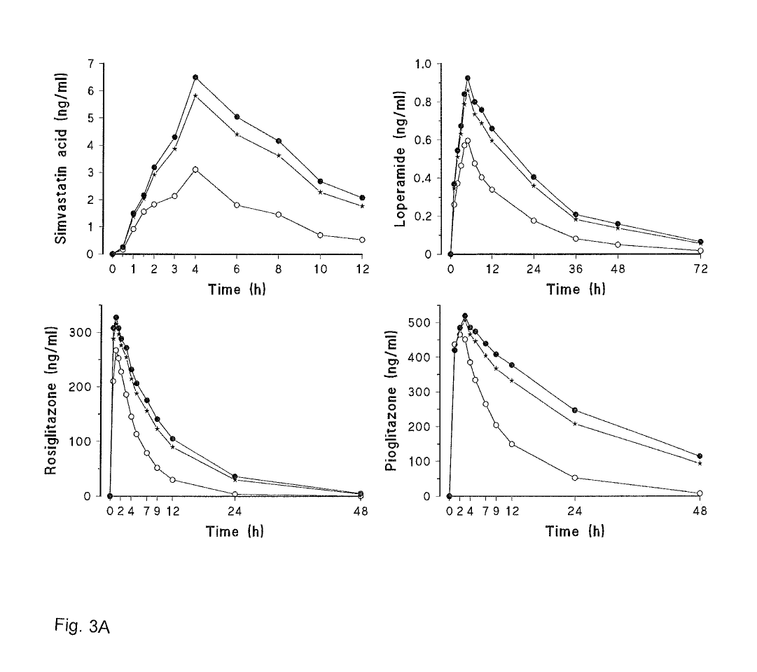

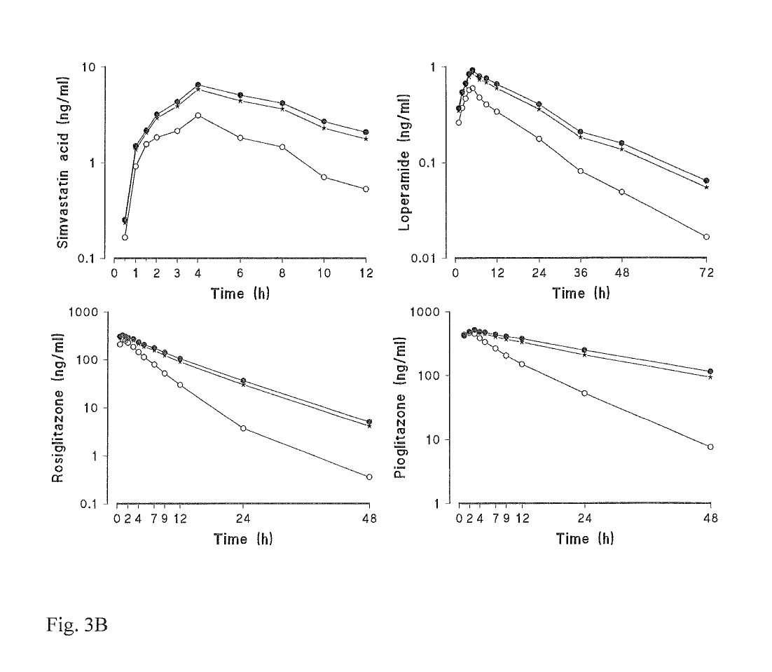

The effect of gemfibrozil (600 mg=black circles, 100 mg=stars, or 0 mg=open circles) given twice daily on plasma concentrations of four different CYP2C8/OATP1B1 substrate drugs (simvastatin 40 mg, loperamide 4 mg, rosiglitazone 4 mg and pioglitazone 15 mg orally). The results are shown in FIG. 3A. FIG. 3B describes the same data on semi logarithmic scale to demonstrate the effect gemfibrozil on elimination phase (half-life).

### Example 4

The effect of various oral single doses (30 mg, 100 mg, 300 mg and 900 mg) of gemfibrozil on the total plasma exposure, i.e., area under the plasma concentration-time curve (AUC), of a CYP2C8 and OATP1B1 substrate drug (repaglinide) was measured. The mean fold increase in the AUC (AUCᵢ/AUC_{c}) of repaglinide with the different gemfibrozil doses in six human subjects is given in relation to the mean average plasma concentration of gemfibrozil 1-O-β-glucuronide during 10 hours after gemfirozil dosing (C_{avg}). The subjects ingested in a cross-over study a small dose (0.25 mg) of repaglinide with different doses of gemfibrozil. Venous blood samples were taken at fixed time points up to 10 hours after gemfibrozil dosing, and plasma repaglinide, gemfibrozil and gemfibrozil 1-O-β-glucuronide concentrations were determined using a validated liquid chromatographic-tandem mass spectrometric (LC/MSMS) method. The data were fitted to the following mechanistic model (equation):

$$\frac{{AUC}_{i}}{{AUC}_{c}} = \frac{1}{\frac{fm , CYP 2 C 8}{( 1 + [ \begin{matrix}kinact / Kl \cdot ( Ch , u / Cp , tot ) \cdot \\ Cavg , 10 h / ke\end{matrix} ] )} + 1 - fm , CYP 2 C 8}$$

$$Ch , u / Cp , tot = 0.282$$

$$fm , CYP 2 C 8 = 88.6 \%$$

The model-based fold increases in AUC, equivalent to 50%, 90% and 95% inhibition of CYP2C8, are indicated by dotted/dashed horizontal lines in FIG. 4. A steep concentration-effect relationship was observed with small gemfibrozil doses, reaching strong (90%) inhibition of the CYP2C8 enzyme already with a dose of 100 mg gemfibrozil. In repeated dosing, the effect of small gemfibrozil doses (<30 mg) increased due to the irreversible inhibition of CYP2C8 and slow synthesis rate (t_{1/2} about 24 h) of new CYP2C8 enzyme.

1.-35. (canceled)

36. A pharmaceutical composition comprising gemfibrozil and a CYP2C8 and/or OATP substrate drug provided that the amount of gemfibrozil is not 200 mg and the amount of atorvastatin is not 40 mg.

37. The pharmaceutical composition of claim 36, wherein the CYP2C8 and/or OATP substrate drug is selected from the group consisting of repaglinide, rosiglitazone, pioglitazone, troglitazone, montelukast, zafirlukast, pranlukast, paclitaxel, all-trans-retinoic acids, amiodarone, amodiaquine, chloroquine, piperaquine, imatinib, nilotinib, lapatinib, diltiazem, verapamil, loperamide, silybin, diclofenac, bexarotene, tazarotenic acid, cyclophosphamide, simvastatin, lovastatin, atorvastatin, cerivastatin, fluvastatin and rosuvastatin.

38. The pharmaceutical composition of claim 36, wherein the amount of gemfibrozil is 0.5 mg-100 mg/dose.

39. The pharmaceutical composition of claim 38, wherein the amount of gemfibrozil is 100 mg and the amount of repaglinide is 0.25 mg.

40. The pharmaceutical composition of claim 38, wherein the amount of gemfibrozil is 30 mg and the amount of repaglinide is 0.25 mg.

41. The pharmaceutical composition of claim 36, wherein the composition is formulated in a sustained release formulation.

42. The pharmaceutical composition of claim 36, wherein the composition is formulated in an instant release formulation.

43. The pharmaceutical composition of claim 36, wherein the composition is not for intrajejunal administration.

44. The pharmaceutical composition of claim 36, wherein the formulation is an oral dosage form.

45. The pharmaceutical composition of claim 36, wherein gemfibrozil and the substrate drug are formulated in the same single formulation.

46. The pharmaceutical composition of claim 36, wherein gemfibrozil is formulated in a first formulation and the CYP2C8 and/or OATP substrate drug is formulated in a second formulation, and the first and the second formulations are administered simultaneously or sequentially to a mammal.

47. A pharmaceutical composition comprising gemfibrozil in amount of less than 300 mg, preferably about 200 mg.

48. The pharmaceutical composition of claim 47, wherein gemfibrozil is the single therapeutically active agent in the composition.

49. A method for adjusting the effect of a CYP2C8 and/or OATP substrate drug in a mammal, comprising administering a CYP2C8 and/or OATP substrate drug to the mammal, and administering gemfibrozil to the mammal in an amount of less than 1 200 mg/day.

50. The method of claim 49, wherein gemfibrozil is administered in an amount of less than 900 mg/day.

51. The method of claim 49 for optimizing plasma profile and/or pharmacokinetics of a CYP2C8 and/or OATP substrate drug.

52. The method of claim 49 for enhancing the therapeutic efficacy of a CYP2C8 and/or OATP substrate drug.

53. The method of claim 49 for preventing adverse and/or side-effects of a CYP2C8 and/or OATP substrate drug.

54. The method of claim 49 for preventing and/or inhibiting the formation of toxic metabolites of a CYP2C8 and/or OATP substrate drug.

55. The method of claim 49, wherein gemfibrozil and the CYP2C8 and/or OATP substrate drug are administered simultaneously or sequentially to a mammal.
